(19) United States
(12) Patent Application Publication
(10) Pub. No.: US 2024/0148512 A1
(43) Pub. Date: May 9, 2024

(54) TIBIA COMPONENT AND KNEE JOINT ENDOPROSTHESIS SYSTEM

(71) Applicant: Aesculap AG, Tuttlingen (DE)

(72) Inventors: Berna Richter, Muehlheim (DE); Martin Nonnenmann, Wurmlingen (DE); Arthur Bollinger, Tuttlingen (DE); Claudia Blender, Renquishausen (DE); Vincent Marie, Engen (DE)

(21) Appl. No.: 18/405,589

(22) Filed: Jan. 5, 2024

Related U.S. Application Data
(63) Continuation of application No. PCT/EP2022/068976, filed on Jul. 7, 2022.

(30) Foreign Application Priority Data
Jul. 8, 2021 (DE) 10 2021 117 733.2

(51) Int. Cl. A61F 2/38

(52) U.S. Cl. CPC A61F 2/389; A61F 2002/3054

(57) ABSTRACT

In order to provide a tibial component for a knee joint endoprosthesis, wherein the tibial component comprises a tibial plate with a top side and a bottom side and comprises an anchoring projection protruding from the bottom side for insertion into the medullary space of a tibia to anchoring the tibial component to the tibia, said tibial component enabling an improved alignment on the tibia, it is proposed that the anchoring projection is mounted on the tibial plate so as to be pivotable and/or rotatable about a longitudinal axis of the anchoring projection.

Furthermore, an improved knee joint endoprosthesis system is proposed.

The present invention relates to a tibial component for a knee joint endoprosthesis, wherein the tibial component comprises a tibial plate with a top side and a bottom side and comprises an anchoring projection protruding from the bottom side for insertion into the medullary space of a tibia for anchoring the tibial component to the tibia, wherein the anchoring projection is mounted on the tibial plate so as to be pivotable and/or rotatable about a longitudinal axis of the anchoring projection, wherein the tibial component comprises a fixing device for fixing the anchoring projection in an implantation position, and wherein the fixing device is movable from an alignment position, in which the anchoring projection and the tibial plate are movable and alignable relative to one another, into the implantation position, in which the anchoring projection and the tibial plate are held against one another so as to be immovable relative to one another.

The present invention further relates to a knee joint endoprosthesis system with at least one femoral component for anchoring to a distal end of a femur and at least one tibial component for anchoring to a proximal end of a tibia, wherein the at least one femoral component and the at least one tibial component are configured corresponding to one another to form a knee joint endoprosthesis.

Knee joint endoprosthesis systems of the kind described at the outset are used to replace damaged knee joints of patients by implanting a knee joint endoprosthesis suitable for the patient. Despite a wide variety of prosthesis types, there is still a non-negligible number of patients who are dissatisfied with the result after implantation of a knee joint endoprosthesis. One problem in particular is the optimal orientation, also referred to as alignment.

A new approach to improving patient satisfaction is so-called kinematic alignment. This is based on a positioning of the knee joint endoprosthesis, taking into account the natural and individual axes of movement of the prearthrotic knee joint. This restores the natural articulation line and leg axis position to their original alignment. Theoretically, this also maintains a natural stable capsule-ligament tension without relieving the ligaments through a so-called “release”. In contrast to this new approach, in a classic alignment of the knee joint endoprostheses, the implant components, in particular the tibial component and the femoral component, are aligned perpendicularly to the mechanical axis.

A tibial component and a knee joint endoprosthesis system of the kind described at the outset are known from DE 20 2007 004 508 U

It is therefore an object of the present invention to provide a tibial component and a knee joint endoprosthesis system of the kind described at the outset, which, in particular, enable a simple handling.

This object is achieved, in accordance with the invention, in a tibial component of the kind described at the outset in that the fixing device comprises a fixing element and in that the second joint element in the implantation position is clampingly held between the fixing element and the first joint element.

The solution proposed in accordance with the invention makes it possible, in particular, to provide tibial components that allow for a desired alignment of the anchoring projection relative to the tibial plate. The anchoring projection can be mounted on the tibial plate, in particular, so as to be steplessly pivotable and/or rotatable. Alternatively, it is also possible, in particular, to predetermine a multitude of defined pivot and rotational positions of the anchoring projection relative to the tibial plate, i.e. a detented pivoting or a detented rotation. These also enable a multitude of different alignments of the anchoring projection relative to the tibial plate, but not in a stepless manner. As a result of the proposed special mounting of the anchoring projection on the tibial plate, it is possible, in particular, to always optimally align the anchoring projection so that it can be inserted anatomically correctly into the medullary space of the tibia of the patient. In particular, a conflict between the anchoring projection and a bone surface of the tibia that delimits the medullary space can thus be prevented. In particular, an anchoring projection of the tibial component can thus be aligned in any manner both in the anterior-posterior direction and medial-lateral direction. Thus, stress peaks, which are caused, in particular, by a pressure or an edge load of the anchoring projection in the medullary space on the inner cortical of the tibia, can be minimized. Due to the described ability to optimally align the anchoring projection of the tibial component, in particular a kinematic alignment of the knee joint endoprosthesis is able to be achieved at all, namely in each patient. In particular, this makes it possible to maintain a natural stable capsule-ligament tension without the need to reduce ligament tension by partially severing ligaments on the patient's knee. This so-called “release” is therefore not necessary. In accordance with the invention, the tibial component comprises a fixing device for fixing the anchoring projection in an implantation position, wherein the fixing device is transferable from an alignment position, in which the anchoring projection and the tibial plate are movable and alignable relative to one another, into the implantation position, in which the anchoring projection and the tibial plate are held against one another so as to be immovable relative to one another. The fixing device thus makes it possible, in particular, to fix the anchoring projection in a desired alignment relative to the tibial component. In the alignment position, the tibial plate and the anchoring projection are mounted or held against one another, but are movable relative to one another so that they can be aligned relative to one another in the desired manner. By transferring the fixing device from the alignment position into the implantation position, a mobility of the anchoring projection and the tibial plate relative to one another is canceled, so that the tibial component can be permanently anchored to the tibia in a defined manner, namely without the anchoring projection and the tibial plate being able to move relative to one another. In the following, the term “fixing position” is used synonymously for the term “implantation position”. It is favorable that the fixing device comprises a fixing element and that the second joint element in the implantation position is clampingly held between the fixing element and the first joint element. Thus, the fixing device forms, in particular, a clamping device for the second joint element, which makes it possible to clampingly fix the second joint element, for example a spherical joint projection, in a hollow-spherical joint receptacle. Such a clamping device enables, in particular, a simple handling of the tibial component during implantation. DE 20 2007 004 508 U1 describes a solution for holding an anchoring projection immovably on the tibial plate in the fixing position that utilizes tensile forces. For this purpose, a tension anchor rotatably mounted on a shaft adapter is used. In contrast, in the proposed further development, the anchoring projection and the tibial plate are held against one another immovably in the fixing position by pressing forces alone. In the fixing position, the fixing element presses the second joint element against the first joint element in a clamping manner. This makes handling simpler, as the anchoring projection does not have to be rotated when transferring the tibial component from the alignment position into the fixing position, unlike in the case of the tibial component described in DE 20 2007 004 508 U1. This makes it possible in the first place to transfer the tibial component from the alignment position into the fixing position and without holding the anchoring projection and a shaft possibly coupled thereto on the distal side, only when the anchoring projection is already inserted into a cavity of the tibia. This simplifies the alignment of the anchoring projection and shortens the time required for implantation.

For a stable anchoring of the tibial component, it is favorable if the anchoring projection is arranged or formed in the region of a symmetry plane of the tibial plate

Preferably, the anchoring projection is arranged or formed in a middle or central region of the bottom side of the tibial plate. In particular, it can thus be securely inserted into and anchored in a central medullary space of the tibia.

It is favorable if the anchoring projection is of rotationally symmetrical configuration or substantially rotationally symmetrical configuration relative to its longitudinal axis. For example, the anchoring projection may be configured in the form of a cylindrical stump or in the form of truncated cone.

It is advantageous if the anchoring projection is mounted on the tibial plate in an articulated manner. In particular, the anchoring projection may be mounted on the tibial plate in a hinge-jointed or ball-jointed manner. Depending on the kind of mounting, an optimal adjustability of the anchoring projection relative to the tibial plate can be achieved. In particular, a ball-jointed mounting of one end of the anchoring projection allows for a free alignment of a free end thereof in a defined angular range relative to a surface normal of the bottom side of the tibial plate and this also over a range of 360° of rotation relative to the surface normal.

In accordance with a preferred embodiment, provision may be made that the tibial component comprises a joint device with a first joint element and a second joint element, that the first joint element is arranged or formed on the tibial plate, that the second joint element is arranged or formed on the anchoring projection, and that the first joint component and the second joint component are in engagement with one another. Such a joint device enables, in particular, a defined pivoting and/or rotation of the joint elements of the joint device that are in engagement with and cooperate with one another. In particular, the joint device may be configured to establish a hinge-jointed or a ball-jointed connection between the joint elements.

Preferably, the first joint component is configured in the form of a joint receptacle and the second joint component in the form of a joint projection engaging into the joint receptacle. A joint device configured in that way can be produced in a simple manner and enables a flexible alignment of the anchoring projection relative to the tibial plate.

It is favorable if the joint projection is of spherical configuration and if the joint receptacle has a hollow-spherical abutment face for the joint projection. This configuration makes it possible in a simple manner to produce a ball-jointed connection with the cooperating joint components. The anchoring projection with a joint ball can thus be rotated in a simple manner by 360° relative to its longitudinal axis in the joint receptacle and also be pivoted relative to a surface normal of the bottom side of the tibial plate, namely preferably within a predetermined angular range. This angular range can be limited, in particular, by suitable stops on the anchoring projection and/or on the tibial plate.

Preferably, the joint receptacle extends away from the bottom side of the tibial plate. This configuration allows, in particular, the formation of the joint receptacle on a projection protruding from the bottom side. As a result, in particular, a maximum angular range of a pivoting of the anchoring projection can be predetermined and maximized in a simple manner.

It is favorable if the joint receptacle is of rotationally symmetrical configuration. In particular, it may be oriented rotationally symmetrically relative to a surface normal of the bottom side of the tibial plate. Such a joint receptacle can be produced in a simple manner and enables a ball-jointed connection of the anchoring projection to the tibial plate.

In particular, in order to be able to mount the anchoring projection on the tibial plate in a simple manner, it is advantageous if a perforation is formed on the tibial plate, said perforation passing from the top side through the first joint element. Thus, for example, with appropriate dimensioning of the anchoring projection, the anchoring projection can be inserted through the perforation coming from the top side of the tibial plate and be passed therethrough.

The tibial component can be configured in a particularly compact manner if the perforation comprises the joint receptacle. In other words, the joint receptacle can be arranged or formed in the region of the perforation.

It is favorable if the anchoring projection is dimensioned in such a way that it is able to be passed from the top side of the tibial plate through the perforation, and if the joint receptacle defines a narrowest point, which forms a stop for the second joint element acting in the proximal direction. This configuration makes it possible, in particular, to position the tibial plate on a prepared tibia of the patent and then to insert the anchoring projection from the top side of the tibial plate through the perforation into the medullary space of the tibia. Here, the anchoring projection can be aligned in the desired manner relative to the tibial plate. In particular, the narrowest point of the joint receptacle prevents the anchoring projection from slipping through the perforation. In other words, a movement of the anchoring projection is thus limited in the distal direction by the correspondingly configured joint receptacle.

In accordance with a further preferred embodiment, provision may be made that laterally next to the anchoring projection at least one stabilization projection is arranged or formed on the bottom side of the tibial plate and pointing away therefrom. In particular, two stabilization projections may be arranged or formed in the manner described. In particular, these may protrude mirror-symmetrically to one another from the bottom side of the tibial plate laterally next to the anchoring projection. In particular, they can stabilize the first joint element of the joint device and protrude laterally therefrom.

It is favorable if the at least one stabilization projection is of rectilinear or curved configuration. In particular, the stabilization projection may be convexly curved in the anterior direction. In this way, in particular, an optimal stabilization of a connection of the tibial component to the tibia can be achieved, in particular a rotation of the tibial plate on the tibia can be easily prevented in this way.

An optimal anchoring of the tibial component to the tibia can be achieved, in particular, by the tibial component comprising a shaft that is connectable to the anchoring projection in a force-locking and/or positive-locking manner. In particular, the shaft can be configured to be screwable to the anchoring projection. This allows a surgeon to optimally stabilize the tibial component on the tibia depending on a size of the patient's medullary canal by selecting a shaft that is adapted to the size of the medullary space, i.e. in particular to its length and cross section. Here, the shaft may be, in particular, of rectilinear configuration or also curved.

The anchoring projection and the shaft can be connected to one another in a simple manner if an externally threaded portion is formed on the anchoring projection, if the shaft has an anchoring projection receptacle for the anchoring projection, and if an internally threaded portion corresponding to the externally threaded portion is formed on the anchoring projection receptacle.

The fixing device can be transferred in a simple manner from the alignment position into the fixing or implantation position if the fixing element comprises a screw element with an external thread and if the perforation commencing from the top side of the tibial plate comprises an internal thread corresponding to the external thread. In this way, the screw element can be arranged on the tibial plate in a defined manner and press the second clamping element against the first clamping element in order to immovably fix it to the tibial plate in the fixing position by exerting a pressing force. The screw element may have, in particular, a tool element receptacle, which points away from the top side of the tibial plate. For example, a tool can thus be used to screw the screw element into the tibial plate in a simple manner.

A compact structure of the tibial component can be achieved, in particular, by the fixing element in the implantation position closing the perforation.

In order to prevent a movement of the second joint element relative to the first joint element in the fixing or implantation position, it is advantageous if the fixing element has a fixing element clamping face, which in the implantation position is clampingly held against the second joint element.

Furthermore, it may be advantageous if the fixing element comprises a clamping element and if the fixing element clamping face is formed on the clamping element. Thus, in particular, optimal material pairings for clamping the second joint element against the first joint element can be selected, independently of the material from which, e.g., the screw element is made. In particular, the clamping element can be formed separately from the screw element or can form a unit together therewith.

The screw element and the clamping element are favorably formed in one piece or as two mutually separate components. A two-part configuration of the screw element and clamping element makes it possible, in particular, to avoid a twisting movement of the clamping element relative to the second joint element when the screw element and the tibial plate are screwed together.

It is favorable if the clamping element has a screw element abutment face, which is configured facing away from the fixing element clamping face and abuts against the screw element in the implantation position. In particular, the screw element abutment face may be of planar configuration A good contact over a surface between the screw element and the clamping element can thus be established in order to press the clamping element with the screw element against the second joint element in a defined manner and thus clampingly hold the second joint element against the first joint element in the fixing position only by exerting a pressing force.

It is advantageous if the perforation has a clamping element receptacle adjoining the joint receptacle in the proximal direction for accommodating the clamping element. A clamping element receptacle arranged in that way makes it possible to accommodate the clamping element in a defined manner and thereby position it on the tibial plate.

It is advantageous if the perforation has a screw element receptacle adjoining the clamping element receptacle in the proximal direction for accommodating the screw element. This also allows the screw element to be positioned in or at the perforation in a defined manner.

A cross sectional area defined by the screw element receptacle is favorably greater than a cross sectional area defined by the clamping element receptacle. Thus, only the clamping element can be inserted into the clamping element receptacle, but not the screw element if the clamping element and the screw element are dimensioned according to the respective receptacles provided therefor. In addition, the configuration of the screw element receptacle and the clamping element receptacle can also limit a maximum movement of the clamping element and the screw element in the distal direction, in particular also a position of the screw element and the clamping element in the fixing or implantation position.

The tibial component can be configured in a simple manner if the top side and/or the bottom side of the tibial plate are of planar or substantially planar configuration.

The object stated at the outset is further achieved, in accordance with the invention, in a knee joint endoprosthesis system of the kind described at the outset in that the at least one tibial component is configured in the form of one of the tibial components described above.

Equipping a knee joint endoprosthesis system with one of the tibial components described above has the advantage, in particular, that an optimally suitable knee joint endoprosthesis for a patient can be formed in order to achieve the kinematic alignment discussed at the outset. With such an optimized alignment of a knee joint replacement, in particular, a satisfaction of the patients after a knee joint endoprosthesis implantation can be significantly improved.

It is advantageous if the knee joint endoprosthesis comprises at least one meniscal component that is coupleable to the tibial component and if the meniscal component has joint face that cooperates with the at least one femoral component. Thus, an optimal sliding pairing between the meniscal component and the femoral component can be formed. In particular, a wide variety of knee joint endoprostheses can thus be equipped with one of the advantageous tibial components described above. The meniscal component can be coupled to the tibial component so as to be either immovable or movable.

In order to enable an optimal adaptation of a knee joint endoprosthesis to a patient, it is favorable if the knee joint endoprosthesis system comprises a plurality of shafts that differ in their length and/or their cross section for selectively coupling to the anchoring projection of the tibial component.

The subsequent description of preferred embodiments of the invention serves in conjunction with the drawings for further explanation. In the drawings:

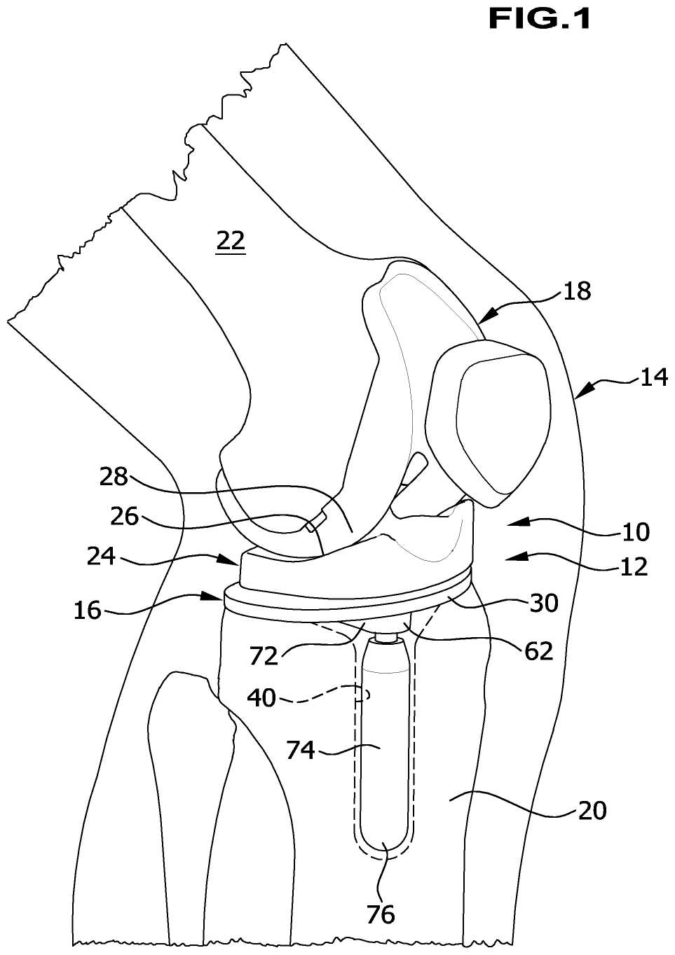

FIG. 1: shows a schematic perspective total view of an embodiment of a knee joint endoprosthesis;

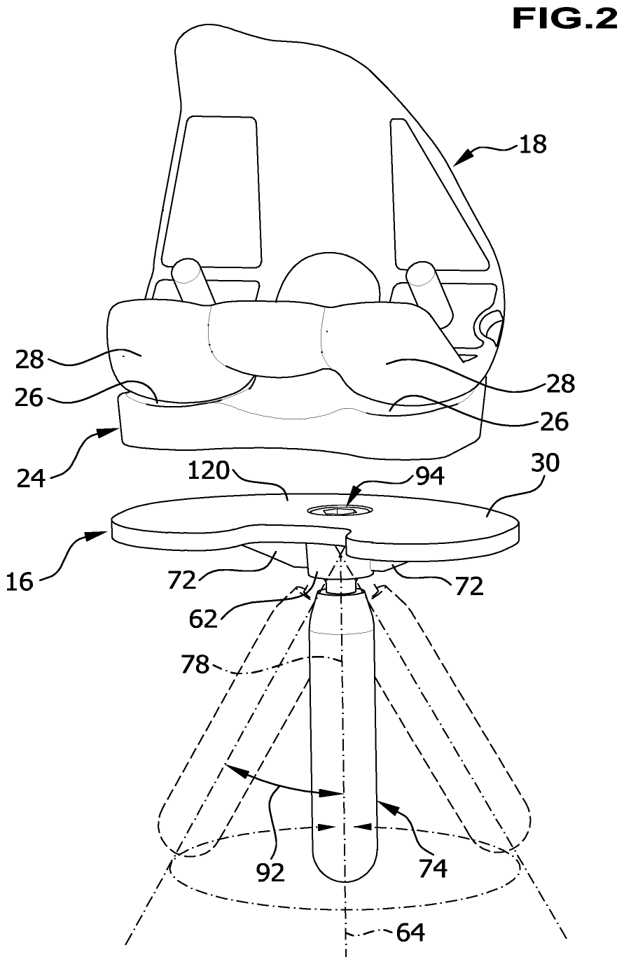

FIG. 2: shows a schematic perspective, partial exploded view of the knee joint endoprosthesis from FIG. 1 with different alignment positions of a shaft of the tibial component drawn schematically;

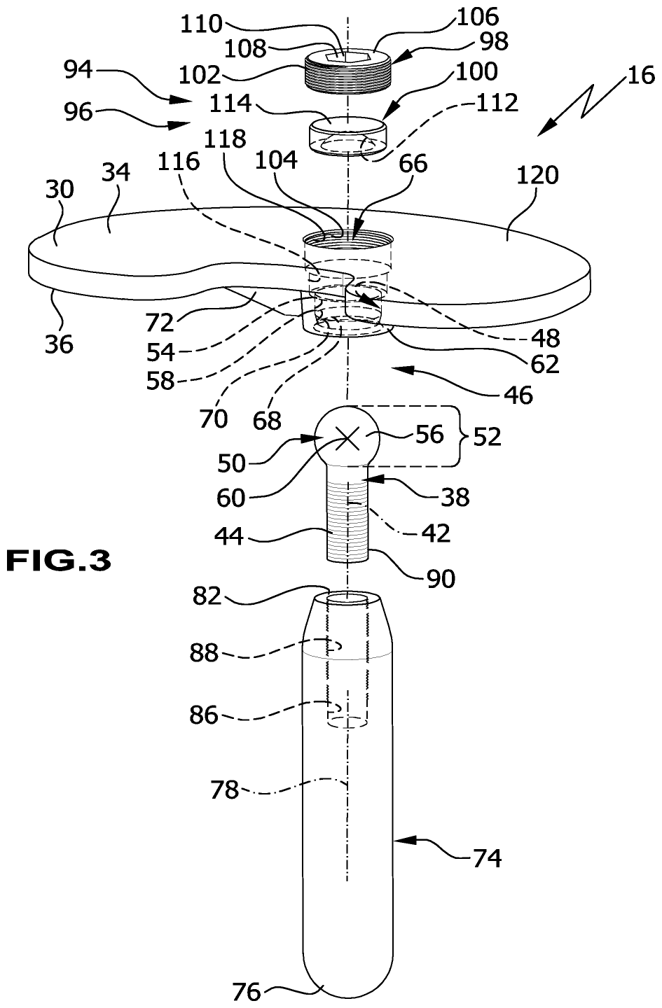

FIG. 3: shows an exploded view of the tibial component from FIGS. 1 and 2;

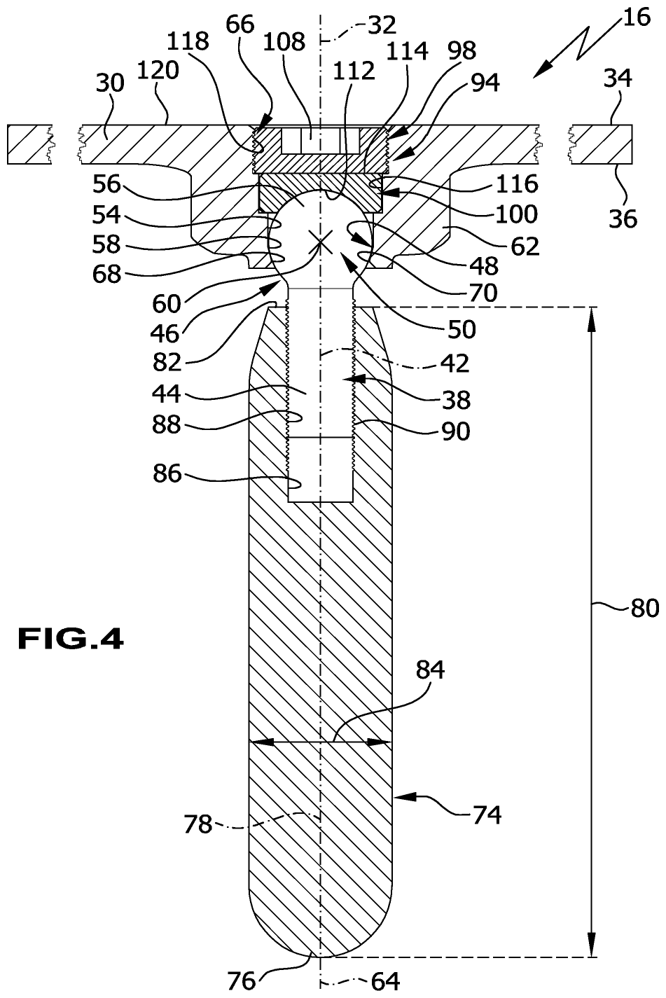

FIG. 4: shows a section view of the tibial component from FIGS. 1 to 3 with a shaft aligned perpendicularly to a top side and bottom side of the tibial component; and

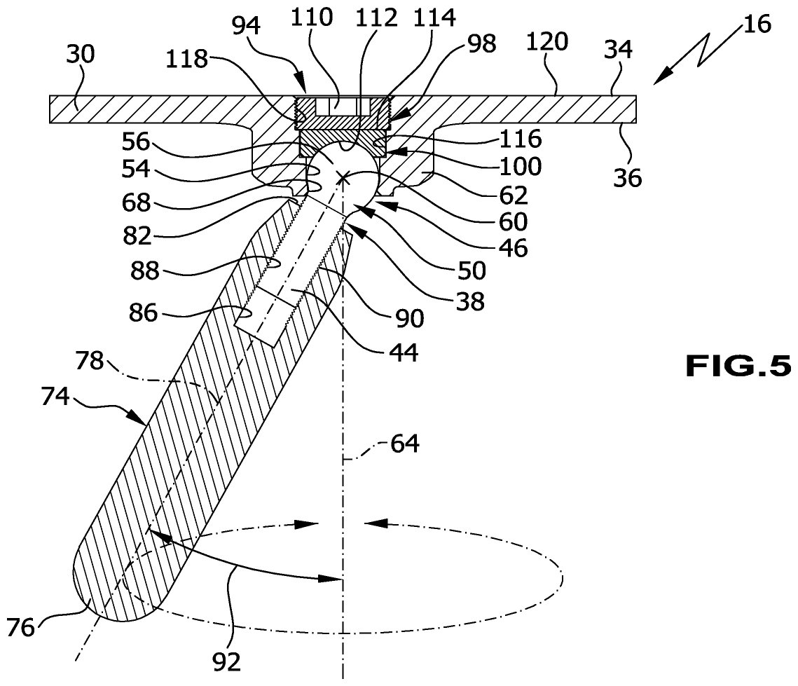

FIG. 5: shows a view analogous to FIG. 4 with an anchoring projection pivoted relative to the tibial plate with a shaft arranged thereon.

A first embodiment of a knee joint endoprosthesis 10 of a knee joint endoprosthesis system 12 is depicted schematically in FIG. 1. The knee joint endoprosthesis 10 is implanted as a replacement for a degenerated natural knee joint in the knee 14 of a patient.

The knee joint endoprosthesis 10 comprises a tibial component 16 and a femoral component 18 that cooperates therewith. The tibial component 16 is configured to anchor to a proximal, prepared end of a tibia 20. The femoral component 18 is configured to anchor to a distal, prepared end of a femur 22. The tibial component 16 and the femoral component 18 are configured corresponding to one another to form the knee joint endoprosthesis 10.

The embodiment of the knee joint endoprosthesis 10 depicted in FIGS. 1 and 2 comprises an optional meniscal component 24, which is arranged between the tibial component 16 and the femoral component 18. In the described embodiment, the meniscal component 24 is selectively able to be fixed to the tibial component 16 or is able to be moved relative thereto.

The meniscal component 24 has a joint face 26 that cooperates with the femoral component 18. This abuts against a condyle face 28 defined by the femoral component 18, wherein the condyle face 28 is able to roll on and/or slide along the joint face 26.

The tibial component 16 comprises a tibial plate 30, which in a plan view is of mirror symmetrical configuration relative to a symmetry plane 32 and is of substantially U-shaped or kidney-shaped configuration. The tibial plate 30 defines a top side 34 and a bottom side 36 pointing in the opposite direction.

An anchoring projection 38 protrudes from the bottom side 36, said anchoring projection being configured to anchor the tibial component in the medullary space 40 of the tibia 20, which is drawn schematically in FIG. 1 with dashed lines.

The anchoring projection 38 is mounted on the tibial plate 30 so as to be pivotable and/or rotatable about a longitudinal axis 42 defined by said anchoring projection.

The anchoring projection 38 is of rotationally symmetrical or substantially rotationally symmetrical configuration relative to the longitudinal axis 42. It comprises a distal end portion 44, which is of cylindrical or substantially cylindrical configuration.

The tibial component 16 comprises a joint device 46 with a first joint element 48 and a second joint element 50. The first joint element 48 and the second joint element 50 are in engagement with one another, in particular partially in positive-locking manner. The first joint element 48 is arranged or formed on the tibial plate 30. The second joint element 50 is arranged or formed on the anchoring projection 38 and defines a proximal end portion 52 thereof.

The first joint element 48 and thus also the anchoring projection 38 are arranged or formed in a region of the symmetry plane 32 of the tibial plate 30. In addition, the first joint element 48 and thus also the anchoring projection 38 are arranged or formed in a middle or central region of the bottom side 36 of the tibial plate 30.

The anchoring projection 38 is mounted on the tibial plate 30 in an articulated manner, in particular in a hinge-jointed or ball-jointed manner, by the joint device 46.

In the described embodiment of the tibial component 16, the first joint component 48 is configured in the form of a joint receptacle 54. The second joint component 50 is configured in the form of a joint projection 56 engaging into the joint receptacle 54. In the embodiment described, the joint projection 56 is of spherical configuration and has a diameter that is greater than a diameter of the distal end portion 44. The joint receptacle 54 defines a hollow-spherical abutment face 58. In this way, the joint projection 56 configured in the form of a joint ball can be rotated in the joint receptacle 54 and pivoted about a center 60 of the joint projection 56.

The joint receptacle 54 extends away from the bottom side 36 of the tibial plate 30 and is formed on a sleeve-shaped projection 62. The joint receptacle 54 is of rotationally symmetrical configuration, namely relative to a surface normal 64 of the bottom side 36 of the tibial plate 30.

A perforation 66 is formed on the tibial plate 30, said perforation passing through the tibial plate 30 from the top side 34, namely through the first joint element 48. Thus, the perforation 66 comprises the joint receptacle 54, which is formed in the region of a distal end of the projection 62.

The anchoring projection 38 is dimensioned or sized in such a way that it is insertable with the distal end portion 44 coming from the top side 34 of the tibial plate 30 into the perforation 66 and therethrough, namely so far that the joint projection 56 comes into abutment at a narrowest point 68 defined by the joint receptacle 54. The narrowest point 68 forms a stop 70 for the second joint element 50 acting in the proximal direction.

Two stabilization projections 72 project from the projection 62 laterally and symmetrically to one another relative to the symmetry plane 32. Like the projection 62, they protrude pointing away from the bottom side 36 of the tibial plate 30. In the embodiment depicted and described, the stabilization projections 72 are of curved configuration, namely convexly curved pointing in the anterior direction. In alternative embodiments, they are of rectilinear configuration.

The stabilization projections 72 can prevent, in particular, a rotation of the tibial plate 30 relative to the tibia 20 about the surface normal 64. They thus form a kind of anti-rotation device for the tibial component 16.

In order to enable a good anchoring of the tibial component 16 to the tibia 20, the anchoring projection 38 is connectable to a shaft 74 in a force-locking and/or positive-locking manner in the embodiment shown in the Figures. The shaft 74 is of substantially elongated cylindrical or substantially cylindrical configuration and has a rounded distal end 76.

The shaft 74 defines a shaft longitudinal axis 78, which coincides with the longitudinal axis 42 when the shaft 74, as schematically depicted in FIGS. 4 and 5, is connected to the anchoring projection 38.

The shaft defines a length 80 from a proximal end to its distal end 76 and a diameter 64 transverse to the shaft longitudinal axis 78.

Commencing from the proximal end 82, an anchoring projection receptacle 86 is formed, which has an internally threaded portion 88. The anchoring projection receptacle 86 is configured in the form of a blind hole and serves to accommodate the distal end portion 44 of the anchoring projection 38. Formed on the distal end portion 44 is an externally threaded portion 90, namely corresponding to the internally threaded portion 88, such that the shaft 74 and the anchoring projection 38 are able to be screwed together.

The anchoring projection 38 can be coupled to the tibial plate 30 as described above by the distal end portion 44 being inserted coming from the top side 34 into the perforation 66 until the joint projection 56 abuts against the abutment face 58. In this position, the joint elements 48 and 50 are movable and thus alignable relative to one another.

In order to fix the anchoring projection 38 in a defined alignment, for example deflected by a deflection angle 92 relative to the surface normal 64, the tibial component 16 comprises a fixing device 94. With this fixing device, the anchoring projection 38 can be fixed in a fixing or implantation position relative to the tibial plate 30, as it is schematically depicted in FIGS. 4 and 5.

The fixing device can be transferred from an alignment position, in which the anchoring projection 38 and the tibial plate 30 are movable and alignable relative to one another, into the implantation position. As mentioned, the anchoring projection 38 and the tibial plate 30 are held against one another in the implantation position so as to be immovable relative to one another.

The fixing device 94 comprises a fixing element 96 with which the second joint element 50 in the fixing position is able to be braced against the first joint element 48. In other words, in the fixing position, the second joint element 50 can thus be clampingly held between the fixing element 96 and the first clamping element 48.

The fixing element 96 depicted in the Figures is formed in two parts and comprises a screw element 98 and a clamping element 100.

The screw element 100 is of cylindrical configuration and has an external thread 102. Corresponding to the external thread 102, an internal thread 104 is formed at the perforation 66 commencing from the top side 34 of the tibial plate 30. In a planar side face 106 of the screw element pointing away from the anchoring projection 38, a tool receptacle 108 is formed, namely configured in the form of a polygonal socket 110.

The screw element 98 can be screwed, as described, to the tibial plate 30 by means of a screw-in tool that engages into the tool receptacle 108, the screw element then closing the perforation 66 in the fixing position.

The fixing element 96 has a fixing element clamping face 112, which in the fixing position clampingly presses against the joint element 50 by exerting exclusively a pressing force. In order to enable an abutment of the fixing element clamping face 112 over a surface against the second joint element 50, the latter being formed by the spherical joint projection 56, the fixing element clamping face 112 is formed hollow-spherically corresponding to the joint projection 56. The fixing element clamping face 112 is thus formed on the clamping element 100.

In alternative embodiments, the screw element 98 and the clamping element 100 are formed in one piece. In particular, the fixing element 96 may be of monolithic configuration.

As can be easily seen in FIG. 3, in the depicted embodiment, the clamping element 100 has a planar screw element abutment face 114. The latter is configured pointing away from the fixing element clamping face 112 and abuts against the screw element 98 in the fixing position.

The joint receptacle 54 is adjoined proximally by a clamping element receptacle 116, which defines a greater diameter than the joint receptacle 54. The clamping element receptacle 116 serves to accommodate and position the clamping element 100.

Furthermore, the perforation 66 has a screw element receptacle 118 adjoining the clamping element receptacle 116 in the proximal direction for accommodating the screw element 98. The internal thread 104 is formed exclusively in the region of the screw element receptacle 118.

A cross-sectional area defined by the screw element receptacle 118 or a diameter thereof is greater than a cross-sectional area defined by the clamping element receptacle 116 or a diameter thereof.

To transfer the fixing device 94 from the alignment position into the implantation position, the screw element 98 is screwed in the direction toward the spherical joint projection 56 until the clamping element 100 presses the joint projection 56 clampingly against the joint receptacle 54 by exerting a pressing force. Conversely, the tibial component 56 or the fixing device 94 can be transferred back from the implantation position into the alignment position by rotating the screw element 98 in the opposite direction and loosening the screw connection slightly, such that the clamping element 100 releases the joint projection 56 somewhat and thus allows for a rotational and pivoting movement of the anchoring projection 38 relative to the tibial plate 30.

In the embodiment depicted in the Figures, the top side 34 and the bottom side 36 of the tibial plate 30 are of planar or substantially planar configuration.

The top side 34 is configured in the form of a tibial joint face 120. It forms a sliding pairing with a bottom side of the meniscal component 24 with a movable mounting of the meniscal component 24 on the tibial component 16.

In order to be able to adapt knee joint endoprostheses 10 individually to the physiology of a patient, the knee joint endoprosthesis system 12 optionally comprises a plurality of shafts 74 that differ in their length 80 and/or their diameter 84 or their cross section, which can be selectively coupled to the anchoring projection 38 of the tibial component 16.

Furthermore the knee joint endoprosthesis system 12 in other embodiments may also comprise a plurality of tibial components 16 that differ in shape and/or, size and a plurality of femoral components 18 that differ in shape and/or size and a plurality of meniscal components 24 that differ in shape and/or size. In this way, optimally suited knee joint endoprostheses 10 can be provided for people of different sizes.

By means of the joint device 46 provided on the described embodiments of the tibial component 16, it is possible to optimally anchor the tibial component 16 to the tibia 20 independently of an orientation of the medullary space of the tibia 20. The described joint device 46 enables a stepless alignment of the anchoring projection with a shaft 74 optionally arranged thereon in order to achieve the kinematic alignment described at the outset. In this way, patient satisfaction after the implantation of the knee joint endoprosthesis can be improved compared to conventional knee joint endoprostheses.

### REFERENCE NUMERAL LIST

10 knee joint endoprosthesis
12 knee joint endoprosthesis system
14 knee
16 tibial component
18 femoral component
20 tibia
22 femur
24 meniscal component
26 joint face
28 condyle face
30 tibial plate
32 symmetry plane
34 top side
36 bottom side
38 anchoring projection
40 medullary space
42 longitudinal axis
44 distal end portion
46 joint device
48 first joint element
50 second joint element
52 proximal end portion
54 joint receptacle
56 joint projection
58 abutment face
60 center
62 projection
64 surface normal
66 perforation
68 narrowest point
70 stop
72 stabilization projection
74 shaft
76 distal end
78 shaft longitudinal axis
80 length
82 proximal end
84 diameter
86 anchoring projection receptacle
88 internally threaded portion
90 externally threaded portion
92 deflection angle
94 fixing device
96 fixing element
98 screw element
100 clamping element
102 external thread
104 internal thread
106 side face
108 tool receptacle
110 internal polygon
112 fixing element clamping face
114 screw element abutment face
116 clamping element receptacle
118 screw element receptacle
120 tibial joint face

1. Tibial component (16) for a knee joint endoprosthesis (10), wherein the tibial component comprises a tibial plate (30) with a top side (34) and a bottom side (36) and comprises an anchoring projection (38) protruding from the bottom side (36) for insertion into the medullary space (40) of a tibia (20) for anchoring the tibial component (16) to the tibia (20), wherein the anchoring projection (38) is mounted on the tibial plate (30) so as to be pivotable and/or rotatable about a longitudinal axis (42) of the anchoring projection (38), wherein the anchoring tibial component (16) comprises a fixing device (94) for fixing the anchoring projection (38) in an implantation position and wherein the fixing device (94) is movable from an alignment position, in which the anchoring projection (38) and the tibial plate (30) are movable and alignable relative to one another, into the implantation position, in which the anchoring projection (38) and the tibial plate (30) are held against one another so as to be immovable relative to one another, characterized in that the fixing device (94) comprises a fixing element (96) and in that the second joint element (50) in the implantation position is clampingly held between the fixing element (96) and the first joint element (48).

2. Tibial component in accordance with claim 1, characterized in that the anchoring projection (38)
a) is arranged or formed in the region of a symmetry plane (32) of the tibial plate (30)
and/or
b) is arranged or formed in a middle or central region of the bottom side (36) of the tibial plate (30)
and/or
c) is of rotationally symmetrical configuration or substantially rotationally symmetrical configuration relative to its longitudinal axis (42)
and/or
d) the anchoring projection (38) is mounted on the tibial plate (30) in an articulated manner, in particular in a hinge-jointed or ball-jointed manner.

3. Tibial component in accordance with any one of the preceding claims, characterized in that the tibial component (16) comprises a joint device (46) with a first joint element (48) and a second joint element (50), in that the first joint element (48) is arranged or formed on the tibial plate (30), in that the second joint element (50) is arranged or formed on the anchoring projection (38), and in that the first joint component (48) and the second joint component (50) are in engagement with one another.

4. Tibial component in accordance with claim 3, characterized in that the first joint component (48) is configured in the form of a joint receptacle (54) and in that the second joint component (50) is configured in the form of a joint projection (56) engaging into the joint receptacle (54).

5. Tibial component in accordance with claim 4, characterized in that
a) the joint projection (56) is of spherical configuration and in that the joint receptacle (54) has a hollow-spherical abutment face (58) for the joint projection (56)
and/or
b) the joint receptacle (54) extends away from the bottom side (36) of the tibial plate (30)
and/or
the joint receptacle (54) is of rotationally symmetrical configuration, in particular relative to a surface normal (64) of the bottom side (36) of the tibial plate (30).

6. Tibial component in accordance with any one of claims 3 to 5, characterized in that a perforation (66) is formed on the tibial plate (30), said perforation passing through the tibial plate (30) from the top side (34) through the first joint element (48),
wherein, in particular,
a) the perforation (66) comprises the joint receptacle (54)
and/or
b) the anchoring projection (38) is dimensioned in such a way that it is able to be passed from the top side (34) of the tibial plate (30) through the perforation (66), and wherein the joint receptacle (54) defines a narrowest point (68), which forms a stop (70) for the second joint element (50) acting in the proximal direction.

7. Tibial component in accordance with any one of the preceding claims, characterized in that laterally next to the anchoring projection (38) at least one stabilization projection (72), in particular two stabilization projections (72), is/are arranged or formed on the bottom side (36) of the tibial plate (30) and pointing away therefrom,
wherein the at least one stabilization projection (72) is of rectilinear or curved configuration, in particular of convexly curved configuration pointing in the anterior direction.

8. Tibial component in accordance with any one of the preceding claims, characterized in that the tibial component (16) comprises a shaft (74), which is connectable in a force-locking and/or positive-locking manner, in particular screwable, to the anchoring projection (38),
wherein, in particular, an externally threaded portion (90) is formed on the anchoring projection (38), wherein the shaft (74) has an anchoring projection receptacle (86) for the anchoring projection (38), and wherein an internally threaded portion (88) corresponding to the externally threaded portion (90) is formed on the anchoring projection receptacle (86).

9. Tibial component in accordance with any one of the preceding claims, characterized in that the fixing element (96)
a) comprises a screw element (98) with an external thread (102), and in that the perforation (66) comprises an internal thread (104) corresponding to the external thread (102) commencing from the top side (34) of the tibial plate (30)
and/or
b) closes the perforation (66) in the implantation position
and/or
c) has a fixing element clamping face (112), which in the implantation position is clampingly held against the second joint element (50).

10. Tibial component in accordance with claim 9, characterized in that the fixing element (96) comprises a clamping element (100) and in that the fixing element clamping face (112) is formed on the clamping element (100).

11. Tibial component in accordance with claim 10, characterized in that the screw element (98) and the clamping element are formed in one piece or as two mutually separate components.

12. Tibial component in accordance with claim 10 or 11, characterized in that the clamping element (100) has a, in particular planar, screw element abutment face (114), which is configured facing away from the fixing element clamping face (112) and abuts against the screw element (98) in the implantation position.

13. Tibial component in accordance with any one of claims 10 to 12, characterized in that the perforation (66) has a clamping element receptacle (116) adjoining the joint receptacle (54) in the proximal direction for accommodating the clamping element (100),
wherein, in particular, the perforation (66) has a screw element receptacle (118) adjoining the clamping element receptacle (116) in the proximal direction for accommodating the screw element (98),
wherein, further in particular, a cross sectional area defined by the screw element receptacle (118) is greater than a cross sectional area defined by the clamping element receptacle (116).

14. Knee joint endoprosthesis system (12) with at least one femoral component (18) for anchoring to a distal end of a femur (22) and at least one tibial component (16) for anchoring to a proximal end of a tibia (20), wherein the at least one femoral component (18) and the at least one tibial component (16) are configured corresponding to one another to form a knee joint endoprosthesis (10), characterized in that the at least one tibial component (16) is configured in the form of a tibial component (16) in accordance with any one of the preceding claims.

15. Knee joint endoprosthesis system in accordance with claim 14, characterized in that
a) the knee joint endoprosthesis (0) comprises at least one meniscal component that is coupleable to the tibial component, and in that the meniscal component (24) has a joint face (26) that cooperates with the at least one femoral component (18),
and/or
b) the knee joint endoprosthesis system (12) comprises a plurality of shafts (74) that differ in their length (80) and/or their cross section for selectively coupling to the anchoring projection (28) of the tibial component (16).
